(19) United States
(12) Patent Application Publication
(10) Pub. No.: US 2006/0286618 A1
(43) Pub. Date: Dec. 21, 2006

(54) AMP Assay

(76) Inventor: Kristian Jansson, Jyvaskyla (FI)

Correspondence Address: Kristian Jansson;Aumakatu 3, FIN-40320, Jyvaskyla, Finland, FI

(21) Appl. No.: 10/546,648

(22) PCT Filed: Feb. 25, 2003
(86) PCT No.: PCT/FI03/00131

§ 371 Date: Aug. 24, 2005

(51) Int. Cl. C12Q 1/66

(52) U.S. Cl. 435/8

(57) ABSTRACT

The invention provides a method for detecting AMP, which method comprises treating a sample with adenylate kinase, nucleoside-diphosphate kinase, and a phosphate donor under conditions such that when AMP is present in the sample ATP is formed; and detecting the ATP so formed, preferably with the light-producing luciferase-luciferin reaction.

This invention relates to the enzymatic detection of the presence or amount of AMP (adenosine 5′-monophosphate), and other analytes related to the production or consumption of AMP.

In an AMP assay described by Brovko et al. (1994) Analytical Biochemistry, 220, 410-414, AMP is converted to ADP with CTP as a phosphate donor in a reaction catalyzed by adenylate kinase. Subsequently, ADP is converted to ATP with phosphoenolpyruvate as a phosphate donor in a reaction catalyzed by pyruvate kinase. This enzyme is known to be inhibited by phosphate and has an absolute requirement for monovalent cations such as potassium or ammonium ions. The ATP formed thereby is detected with the light-producing luciferase-luciferin reaction. In the presence of ATP and O₂, luciferase catalyzes the oxidation of luciferin, producing light that can be quantitated with a luminometer. Additional products of the reaction are AMP, pyrophosphate, and oxyluciferin. For improved sensitivity, the three enzymes of the assay are co-immobilized onto cyanogen bromide-activated agarose. Nevertheless, the reported detection limit for this assay is as high as 15 pmol.

In an AMP assay described in U.S. Pat. No. 5,891,659, AMP is converted to ATP with phosphoenolpyruvate and pyrophosphate as phosphate donors in a reaction catalyzed by pyruvate phosphate dikinase. This reaction is coupled to the light-producing luciferase-luciferin reaction. However, the two reactions are incompatible in that pyrophosphate is an inhibitor of luciferase activity. Furthermore, the pH optimal for the activity of pyruvate phosphate dikinase is suboptimal for luciferase activity. Therefore, a high concentration of luciferase is required, preferably at least 500 μg/ml, which makes the assay expensive. Pyruvate phosphate dikinase has an absolute requirement for monovalent cations such as potassium or ammonium ions.

In U.S. Pat. No. 6,335,162, AMP is detected by converting it to ATP with phosphoribosylpyrophosphate as a pyrophosphate donor in a reaction catalyzed by phosphoribosylpyrophosphate synthetase. The ATP produced is detected with the light-producing luciferase-luciferin reaction. However, the phosphoribosylpyrophosphate synthetase reaction proceeds to the direction of ATP synthesis only under narrowly defined reaction conditions not compatible with the subsequent luciferase reaction.

Therefore, it is an object of the present invention to provide an assay for AMP, which does not suffer from the disadvantages of the known procedures which make their use difficult or expensive.

Thus, the invention in one embodiment provides a method for detecting AMP, which method comprises treating a sample with adenylate kinase, nucleoside-diphosphate kinase, and a phosphate donor under conditions such that when AMP is present in the sample ATP is formed; and detetecting the ATP so formed, preferably with the light-producing luciferase-luciferin reaction.

In another embodiment, the invention provides a reagent for detecting AMP, which reagent comprises adenylate kinase, nucleoside-diphosphate kinase, and a phosphate donor, and which preferably further comprises luciferase and luciferin.

In yet another embodiment, the invention provides a kit for detecting AMP, which kit comprises in a packaged combination adenylate kinase, nucleoside-diphosphate kinase, and a phosphate donor, and which optionally further comprises luciferase and luciferin.

Appropriate conditions for the method of the present invention include appropriate component concentrations, solution temperature, ionic strength, and incubation time. Such conditions also include the presence of any appropriate additional substances, such as enzyme cofactors, stabilizers, and buffering agents. Appropriate incubation conditions for a given enzyme, or coupled enzyme system, are generally known in the art or are readily determined using standard methods known in the art.

In preferred embodiments the phosphate donor is a nucleoside triphosphate or an analogue thereof, and in particularly preferred embodiments, the phosphate donor is dCTP (2′-deoxycytidine 5′-triphosphate) available for example from Amersham Biosciences (Piscataway, N.J.).

In some embodiments, the ATP formed is detected with a detection system other than the luciferase-luciferin reaction. In a commercially available ATP kit (#366-A, Sigma, St. Louis, Mo.), a combination of two enzymes, phosphoglycerate kinase and glyceraldehyde phosphate dehydrogenase, are used to catalyze the formation of NAD from NADH in the presence of the ATP formed, which is detected as a decrease in NADH-dependent UV absorbance (340 nm) or fluorescence emission (460 nm), as described further in U.S. Pat. No. 6,335,162, incorporated herein by reference. Another detection system for the ATP formed is based on the use of a FAD synthetase-active system in conjunction with FMN to generate FAD. This detection system is fully described in U.S. Pat. No. 4,806,415, incorporated herein by reference.

In preferred embodiments, the ATP formed is detected by the luciferase-luciferin reaction. In the presence of ATP and O₂, luciferase catalyzes the oxidation of luciferin, producing light. Additional products of the reaction are AMP, pyrophosphate and oxyluciferin. The light can be detected by a luminometer or similar light-sensitive instrument, or more specifically, by a photomultiplier, a photodiode, a charged coupled device (CCD), or the like.

Preferred luciferase is recombinant firefly luciferase available for example from Promega (Madison, Wis.) and, as a kit component, from Molecular Probes (Eugene, Oreg.).

By detecting is meant detecting the presence or amount.

By adenylate kinase is meant the enzyme of EC 2.7.4.3, or any enzyme, ribozyme, or the like, capable of catalyzing the conversion of AMP to ADP with a nucleoside triphosphate as a phosphate donor. In the present invention, the preferred adenylate kinase is that of EC 2.7.4.3, and in particular, myokinase from chicken muscle available from Sigma (St. Louis, Mo.). In an application that requires incubation at an elevated temperature, it may be more preferable to use a thermostable adenylate kinase such as myokinase from Bacillus stearothermophilus also available from Sigma.

By nucleoside-diphosphate kinase is meant the enzyme of EC 2.7.4.6, or any enzyme, ribozyme, or the like, capable of catalyzing the conversion of ADP to ATP with a nucleoside triphosphate as a phosphate donor. In the present invention, the preferred nucleoside-diphosphate kinase is that of EC 2.7.4.6, and in particular, Saccharomyces cerevisiaenucleoside-diphosphate kinase available from Sigma. In an application that requires incubation at an elevated temperature, it may be more preferable to use a thermostable nucleoside-diphosphate kinase such as that from Pyrococcus furiosus, described in the published U.S. Patent Application 20010031470, incorporated herein by reference.

In addition to AMP, this invention also relates to the enzymatic detection of other analytes related to the production or consumption of AMP.

For example, RNA in a sample is detected by adding to the sample a ribonuclease that hydrolyzes RNA to nucleoside monophosphates including AMP that is then detected by the method of the present invention. On the other hand, a ribonuclease in a sample is detected by its ability to hydrolyze added RNA to nucleoside monophosphates including AMP that is then detected by the method of the present invention.

The method for detecting AMP of the present invention may be based on the following reactions, wherein dCTP as a phosphate donor may be replaced by another nucleoside triphosphate or an analogue thereby:

AMP+dCTP→ADP+dCDP, catalyzed by adenylate kinase;

ADP+dCTP→ATP+dCDP, catalyzed by nucleoside-diphosphate kinase; and

ATP+O₂+luciferin AMP+pyrophosphate+CO₂+oxyluciferin+light, catalyzed by luciferase.

A preferred phosphate donor such as dCTP is substrate for both adenylate kinase and nucleoside-diphosphate kinase, but may not be substrate for luciferase.

The three reactions shown above are preferably coupled together, such that the ATP consumed in the light-producing luciferase-luciferin reaction is regenerated in the reactions catalyzed by adenylate kinase and nucleoside-diphosphate kinase.

Thereafter, these three reactions occur simultaneously, continuously and repeatedly.

It is confirmed that under appropriate conditions, the intensity of light produced by the above coupled reaction system is substantially constant for at least 30 minutes.

However, in the above coupled reaction system, pyrophosphate will accumulate in the course of time, eventually leading to the inhibition of light production.

Therefore, appropriate conditions for the method of the present invention may include an agent to break down any pyrophosphate generated. Preferably said agent is inorganic pyrophosphatase (EC 3.6.1.1) such as that from Saccharomyces cerevisiaeavailable from Sigma (St. Louis, Mo.).

The following examples are intended to illustrate the present invention and in no way limit any aspect of the invention.

### EXAMPLE 1

Reaction Buffer:

25 mM aqueous Tricine buffer, pH 7.8, 5 mM MgSO₄, 0.1 mM EDTA, and

0.1 mM sodium azide (Molecular Probes, Component E of A-22066, Lot 64B1-1).

Reagent A:

200 μM dCTP (sodium salt, Amersham Biosciences, 272062, Lot 8620),

20 units/ml myokinase (Sigma, M5520, Lot 012K7485), and

20 units/ml nucleoside-diphosphate kinase (Sigma, NO₃₇₉, Lot 119H7455) in Reaction Buffer.

Reagent B:

5 mM D-luciferin (sodium salt, Molecular Probes, Component A of A-22066, Lot 64B1-1), 50 μg/ml luciferase, firefly recombinant (Molecular Probes, Component B of A-22066, Lot 64B1-1), and

10 mM dithiothreitol (DTT) (Molecular Probes, Component C of A-22066, Lot 64B1-1) in Reaction Buffer.

Reagent C:

45 volumes of Reagent A, and

10 volumes of Reagent B.

### EXAMPLE 2

A sample containing 10 pmol AMP (sodium salt, Sigma, A1752, Lot 042K7000) in 45 μl of Reaction Buffer was mixed in a polystyrene test tube (Sarstedt, 55476) with 45 μl of Reagent A in which 200 μM dCTP was replaced with either 500 μM AMPCPP (α, β-methyleneadenosine 5′-triphosphate lithium salt, Sigma, M6517, Lot 101K7028), 500 μM dGTP, 500 μM dCTP or 500 μM dTTP (sodium salts, Roche, Mannheim, Germany, 1969064, Lot 90704020). After 30 min of incubation at room temperature, 10 μl of Reagent B was added, and after 15 min, the tube was read in a Berthold 9509 luminometer for 10 s. RLU=relative light units. Appropriate controls (no AMP, no phosphate donor) were included. The readings for duplicate reactions (I and II) are shown in Table 1.

TABLE 1

| AMP (pmol) | Phosphate donor | RLU I | RLU II | Average RLU |
|---|---|---|---|---|
| — | — | 163 | 166 | 164 |
| 10 | — | 172 | 169 | 170 |
| — | AMPCPP | 374 | 368 | 371 |
| 10 | AMPCPP | 18362 | 19805 | 19084 |
| — | dGTP | 2140 | 1957 | 2048 |
| 10 | dGTP | 54745 | 61576 | 58160 |
| — | dCTP | 690 | 698 | 694 |
| 10 | dCTP | 59787 | 56687 | 58237 |
| — | dTTP | 9278 | 10565 | 9922 |
| 10 | dTTP | 65304 | 60376 | 62840 |

Table 1 shows that a nucleoside triphosphate or an analogue thereof can serve as a phosphate donor for detecting AMP in the present invention. Of the phosphate donors tested, dCTP has the highest signal-to-noise ratio.

### EXAMPLE 3

A sample containing 0.1-10 pmol AMP (sodium salt, Sigma, A1752, Lot 042K7000) or 10 pmol ATP (disodium salt, Molecular Probes, Component D of A-22066, Lot 64B1-1) in 45 μl of Reaction Buffer was mixed in a polystyrene test tube (Sarstedt, 55476) with 45 μl of Reagent A. After 30 min of incubation at room temperature, 10 μl of Reagent B was added, and after 15 s, 15 min and 30 min, the tube was read in a Berthold 9509 luminometer for 10 s. RLU=relative light units. No-analyte control (blank) was included. The average readings for duplicate reactions are shown in Table 2.

TABLE 2

| | Average RLU | | |
|---|---|---|---|
| AMP (pmol) | 15 s | 15 min | 30 min |
| — | 726 | 666 | 646 |
| 0.1 | 1360 | 1287 | 1280 |
| 0.5 | 3684 | 3543 | 3309 |
| 1 | 6938 | 6367 | 6192 |
| 5 | 31993 | 27954 | 26668 |
| 10 | 63780 | 53392 | 51614 |
| ATP (10 pmol) | 63199 | 57107 | 51788 |

Table 2 shows that Reagent A efficiently converts AMP present in a sample to ATP, which is subsequently detected with Reagent B. The light signal is directly proportional to the quantity of AMP and substantially constant for at least 30 minutes. With the method of the invention, fmol quantities of AMP can be detected.

### EXAMPLE 4

A sample containing 0.1-10 pmol AMP (sodium salt, Sigma, A1 752, Lot 042K7000) or 10 pmol ATP (disodium salt, Molecular Probes, Component D of A-22066, Lot 64B1-1) in 45 μl of Reaction Buffer was mixed in a polystyrene test tube (Sarstedt, 55476) with 55 μl of Reagent C, and after 10, 20 and 30 min at room temperature, the tube was read in a Berthold 9509 luminometer for 10 s. RLU=relative light units. No-analyte control (blank) was included. The average readings for duplicate reactions are shown in Table 3.

TABLE 3

| | Average RLU | | |
|---|---|---|---|
| AMP (pmol) | 10 min | 20 min | 30 min |
| — | 512 | 510 | 500 |
| 0.1 | 900 | 890 | 878 |
| 0.5 | 2460 | 2274 | 2004 |
| 1 | 4616 | 4396 | 3772 |
| 5 | 17802 | 17814 | 16730 |
| 10 | 34874 | 33200 | 32031 |
| ATP (10 pmol) | 35456 | 33659 | 31268 |

Table 3 shows that AMP in a sample can be detected in a single step with Reagent C. The light signal is directly proportional to the quantity of AMP and substantially constant for at least 30 minutes.

### EXAMPLE 5

A sample containing 0.1-10 pmol AMP (sodium salt, Sigma, A 1752, Lot 042K7000) or 10 pmol ATP (disodium salt, Molecular Probes, Component D of A-22066, Lot 64B1-1) in 45 μl of Reaction Buffer was mixed in a polystyrene test tube (Sarstedt, 55476) with 55 μl of Reagent C, and after 1, 2, 3, 4 and 5 min at room temperature, the tube was read in a Berthold 9509 luminometer for 10 s. RLU=relative light units. No-analyte control (blank) was included. The average readings for duplicate reactions are shown in Table 4.

TABLE 4

| | Average RLU | | | | |
|---|---|---|---|---|---|
| AMP (pmol) | 1 min | 2 min | 3 min | 4 min | 5 min |
| — | 508 | 504 | 488 | 486 | 489 |
| 0.1 | 866 | 860 | 871 | 848 | 860 |
| 1 | 3844 | 4184 | 4182 | 4144 | 4103 |
| 10 | 34336 | 37216 | 37166 | 37118 | 36792 |
| ATP (10 pmol) | 37374 | 35982 | 35849 | 35014 | 34700 |

Table 4 shows that AMP in a sample can be detected in a single step almost immediately after adding Reagent C. The light signal is directly proportional to the quantity of AMP.

1. A method for detecting AMP, characterized in that it comprises (a) treating a sample with adenylate kinase, nucleoside-diphosphate kinase, and a phosphate donor under conditions such that when AMP is present in the sample, ATP is formed; and (b) detetecting the ATP formed.

2. A method according to claim 1, wherein the ATP formed is detected with the light-producing luciferase-luciferin reaction.

3. A method according to claim 1 or 2, wherein said phosphate donor is a nucleoside triphosphate or an analogue thereof.

4. A method according to claim 1 or 2 wherein said phosphate donor is dCTP.

5. A reagent for detecting AMP, characterized in that it comprises adenylate kinase, nucleoside-diphosphate kinase, and a phosphate donor.

6. A reagent according to claim 5 further comprising luciferase and luciferin.

7. A reagent according to claim 5 or 6, wherein said phosphate donor is a nucleoside triphosphate or an analogue thereof.

8. A reagent according to claim 5 or 6 wherein said phosphate donor is dCTP.

9. A kit for detecting AMP, characterized in that it comprises in a packaged combination adenylate kinase, nucleoside-diphosphate kinase, and a phosphate donor.

10. A kit according to claim 9 further comprising luciferase and luciferin.

11. A kit according to claim 9 or 10, wherein said phosphate donor is a nucleoside triphosphate or an analogue thereof 12. A kit according to claim 9 or 10, wherein said phosphate donor is dCTP.
